(19) United States
(12) Patent Application Publication
(10) Pub. No.: US 2016/0168621 A1
(43) Pub. Date: Jun. 16, 2016

(54) METHODS AND RELATED DEVICES FOR SINGLE MOLECULE WHOLE GENOME ANALYSIS

(71) Applicant: BioNano Genomics, Inc., San Diego, CA (US)

(72) Inventors: Ming Xiao, Huntington Valley, PA (US); Han Cao, San Diego, CA (US)

(21) Appl. No.: 14/881,556

(22) Filed: Oct. 13, 2015

Related U.S. Application Data
(63) Continuation of application No. 13/710,180, filed on Dec. 10, 2012, now abandoned.
Application No. 13/710,180 is a continuation-in-part of application No. 13/001,697, filed on Mar. 22, 2011, now Pat. No. 8,628,919.
Application No. 13/710,180 is a continuation of application No. 13/503,307, filed on May 25, 2012, now abandoned.
(60) Provisional application No. 61/076,785, filed on Jun. 30, 2008.
(60) Provisional application No. 61/253,639, filed on Oct. 21, 2009.

(51) Int. Cl. C12Q 1/68

(52) U.S. Cl. CPC C12Q 1/683

(57) ABSTRACT

Provided are methods of labeling and analyzing features along at least one macromolecule such as a linear biopolymer, including methods of mapping the distribution and frequency of specific sequence motifs or the chemical or proteomic modification state of such sequence motifs along individual unfolded nucleic acid molecules. The present invention also provides methods of identifying signature patterns of sequence or epigenetic variations along such labeled macromolecules for direct massive parallel single molecule level analysis. The present invention also provides systems suitable for high throughput analysis of such labeled macromolecules.

### CROSS-REFERENCE TO RELATED APPLICATIONS

This application is a continuation of U.S. patent application Ser. No. 13/710,180, filed on Dec. 10, 2012, which is a continuation of U.S. patent application Ser. No. 13/503,307, filed on May 25, 2012, which is in turn a 35 U.S.C. §371 application of PCT/US2010/053513, filed Oct. 21, 2010, which is a non-provisional of and claims priority to U.S. Application Ser. No. 61/253,639, filed Oct. 21, 2009, all four applications entitled “METHODS AND RELATED DEVICES FOR SINGLE MOLECULE WHOLE GENOME ANALYSIS.” The present application is also a continuation-in-part of U.S. patent application Ser. No. 13/001,697, filed on Mar. 22, 2011, which is a 371 application of PCT/US2009/049244, filed on Jun. 30, 2009, which is a non-provisional of and claims priority to 61/076,785, filed Jun. 30, 2008, entitled, “Single Molecule Whole Genome Analysis.” All of the foregoing applications are hereby incorporated by reference in their entireties.

### REFERENCE TO SEQUENCE LISTING

A Sequence Listing submitted as an ASCII text file via EFS-Web is hereby incorporated by reference in accordance with 35 U.S.C. §1.52(e). The name of the ASCII text file for the Sequence Listing is SEQLISTING.TXT, the date of creation of the ASCII text file is Mar. 7, 2013, and the size of the ASCII text file is 2 KB.

### TECHNICAL FIELD

The present invention relates to the field of nanotechnology and to the field of single molecule genomic analysis.

### BACKGROUND

Macromolecules, such as DNA or RNA, are long polymer chains composed of nucleotides, whose linear sequence is directly related to the genomic and post-genomic gene expression information of the source organism.

Direct sequencing and mapping of sequence regions, motifs, and functional units such as open reading frames (ORFs), untranslated regions (UTRs), exons, introns, protein factor binding sites, epigenomic sites such as CpG clusters, microRNA sites, transposons, reverse transposons and other structural and functional units are important in assessing of the genomic composition and “health profile” of individuals.

In some cases, the complex rearrangement of the nucleotides' sequence, including segmental duplications, insertions, deletions, inversions and translocations, during an individual's life span leads to disease states including genetic abnormalities or cell malignancy. In other cases, sequence differences, copy number variations (CNVs), and other differences between different individuals' genetic makeup reflects the diversity of the genetic makeup of the population and differential responses to environmental stimuli and other external influences, such as drug treatments.

Other ongoing processes such as DNA methylation, histone modification, chromatin folding, and other changes that modify DNA-DNA, DNA-RNA or DNA-protein interactions influence gene regulations, expressions and ultimately cellular functions resulting in diseases and cancer.

Genomic structural variations (SVs) are much widespread, even among healthy individuals. The importance to human health of understanding genome sequence information has become increasingly apparent.

Conventional cytogenetic methods such as karyotyping, FISH (Fluorescent in situ Hybridization) provided a global view of the genomic composition in as few as a single cell. These methods reveal gross changes of the genome such as aneuploidy, gain, loss or rearrangements of large fragments of thousands and millions of base pairs. However, these methods suffer from relatively low sensitivity and resolution in detecting medium to small sequence motifs or lesions, as well as being laborious, of limited speed and inconsistent accuracy.

More recent methods for detecting sequence regions, sequence motifs of interests and SVs, such as aCGH (array Comparative Genomic Hybridization), fiberFISH, or massive pair-end sequencing have improved resolution and throughput. These more recent methods are still either indirect, laborious and inconsistent, expensive, and often have limited fixed resolution, providing either inferred positional information relying on mapping back to reference genome for reassembly or comparative intensity ratio information that does not reveal balanced lesion events such as inversions or translocations.

Functional units and common structural variations are thought to encompass from tens of bases to more than megabases. Thus, a method of revealing sequence information and SVs across the resolution scale from sub-kbs (i.e., less than about one kilobase in length) to megabases along large native genomic molecules would be highly desirable in sequencing and fine-scale mapping projects of more individuals in order to catalog previously uncharacterized genomic features.

Furthermore, phenotypical polymorphism or disease states of biological systems, particularly in multiploid organisms such as humans, are consequences of the interplay between the two haploid genomes inherited from maternal and paternal lineage. Cancer is often the result of the loss of heterozygosity among diploid chromosomal lesions.

Current sequencing analysis approaches are largely based on samples derived from averaged multiploidy genomic materials with limited haplotype information. This is largely due to existing front end sample preparation methods currently employed to extract the mixed diploid genomic material from a heterogeneous cell population and then shredding them into random smaller pieces. This approach, however, destroys the native structural information of the diploid genome.

Recently developed second-generation sequencing methods, while having improved throughput, further complicate the delineation of complex genomic information due to more difficult assembly from much shorter sequencing reads.

In general, short reads are harder to align uniquely within complex genomes, additional sequence information is needed to decipher the linear order of the short target region. The order of 25 fold sequencing coverage is needed to reach similar assembly confidence instead of 8-10 fold coverage needed in conventional BAC and shot gun Sanger sequencing (Wendl M C, Wilson R K Aspects of coverage in medical DNA sequencing, BMC Bioinformatics 2008 May 16; 9:239). This imposes further challenges sequencing cost reduction and defeats the original primary goal of dramatically reducing sequencing cost below the target $1000 mark.

Single molecule level analysis of large intact genomic molecules provides the possibility of preserving the accurate native genomic structures by fine mapping the sequence motifs in situ without clonal process or amplification. The larger the genomic fragments are, the less complex the sample population in genomic analytes. In an ideal scenario, only 46 chromosomal fragments need to be analyzed at single molecule level to cover the entire diploid human genome; the sequence derived from such approach has intact haplotype information by its nature.

At a practical level, megabase genomic fragments can be extracted from cells and preserved for direct analysis. This would reduce the burden of complex algorithm and assembly, and also co-relates genomic and/or epigenomic information in its original context more directly to individual cellular phenotypes.

Macromolecules such as genomic DNA are often in the form of semi-flexible worm-like polymeric chains. These macromolecules are normally assumed to have a random coil configuration in free solution. For unmodified dsDNA in biological solution, the persistence length (a parameter defining its rigidity) is typically about 50 nm

In order to achieve the consistent separation of the marked features along large intact macromolecules for quantitative measurements, one approach is to stretch such polymeric molecules in consistent linear form, either on flat surface, chemically or topologically predefined surface patterns, preferably long nanotracks or confined micro/nanochannels.

Methods of stretching and elongate long genomic molecules have been demonstrated, either by using external force such as optical tweezers, liquid-air boundary convective flows (combing), or laminar fluidic hydrodynamic flow.

Elongated forms of molecules will be either stabilized transiently as long as the external force was maintained or more permanently by attaching to a surface enhanced via modification with electrostatic or chemical treatment. Demonstrated elongation of polymeric macromolecules inside micro/nanochannels has been demonstrated by physical entropic confinement (see Cao et al., Applied Phys. Lett. 2002a, Cao et al Applied Phys. Lett. 2002b; U.S. patent application Ser. No. 10/484,293, incorporated herein by reference in their entireries).

Nanochannels with diameters around 100 nm have been shown to linearize dsDNA genomic fragments up to several hundred kilobases to megabases (Tegenfeldt et al., Proc. Natl. Acad. Sci. 2004). Semi-flexible target molecules elongated with nanofluidics can be suspended in a buffer condition within biological range of ion concentration or pH value, hence it is more amenable to perform biological functional assays on such molecules. This form of elongation is also relatively easier for manipulation such as moving charged nucleic acid molecules in electric field or pressure gradient in a wide range of speed from high velocity to complete stationery state with precisely controlled manner.

Furthermore, the nature of fluidic flow in a nanoscale environment precludes turbulence and many of the shear forces that might otherwise fragment long DNA molecules. This is especially valuable for macromolecule linear analysis, especially in sequencing applications in which ss-DNA could be used. Ultimately, the effective read length can be only as long as the largest intact fragment that can be maintained.

In addition to genomics, the field of epigenomics has been recognized as being of singular importance for its roles in human diseases such as cancer. With the accumulation of knowledge in both genomics and epigenomics, a major challenge is understanding how genomic and epigenomic factors correlate directly or indirectly to polymorphism or pathophysiological conditions in human diseases and malignancies.

Whole genome analysis concept has evolved from a compartmentalized approach in which areas of genomic sequencing, epigenetic methylation analysis and functional genomics were studied largely in isolation, to a more multi-faceted holistic approach. DNA sequencing, structural variations mapping, CpG island methylation patterns, histone modifications, nucleosomal remodeling, microRNA function and transcription profiling have been viewed in a more systematic way. However, technologies examining each of above aspects of the molecular state of the cells are often isolated, tedious and non-compatible, which severely complicates a system biology analysis that requires coherent experimental data results.

Single molecule level analysis of large intact native biological samples could provide the potential of studying genomic and epigenomic information of the target samples in true meaningful wholesome analytical way such as overlaying the sequence structural variations with aberrant methylation patterns, microRNA silencing sites and other functional molecular information. (See, e.g., PCT patent application US2009/049244, the entirety of which is incorporated herein by reference.) It would provide a very powerful tool in understanding the molecular functions of cell and diseases genesis mechanism in personalized medicine.

### SUMMARY

The present invention relates, in one aspect, to methods of labeling and analyzing marked features along at least one macromolecule such as a linear biopolymer. The methods, in some embodiments, relate to methods of mapping the distribution and frequency of specific sequence motifs (i.e., pattern, theme) or chemical or proteomic modification state of such sequence motifs along individual unfolded nucleic acid molecules, depending on the length, and sequence of the motif.

Also disclosed are fluidic chips and systems suitable for sorting and linearly unfolding labeled macromolecules. These chips and systems are capable of operating in parallel fashion for optical and non-optical signal analysis.

Another aspect of the invention is identifying double stranded DNA molecules by mapping the distribution of short sequence motifs along the DNA backbone. This provides high spatial resolutions between sequence motifs. Based on this high resolution map, the sequencing reaction was initialized at each of the sequence specific motif sites, and cycled through time to obtain multiple base information at known spatial location, which can be termed STS, or spatial and temporal sequencing. The present invention also relates to the uses of such labeling processes and features.

In one embodiment, marked specific sequence motifs on double stranded DNA are created by nicking single strands of DNA and forming gaps (this may be accomplished by enzymes). The user may then apply a polymerase for strand extension while generating “peeled” short sequence segments called “flaps” simultaneously. These peeled single stranded flaps create available regions for sequence specific hybridization with labeled probes. In some embodiments, bases (including labeled bases or labeled probes) bind to the peeled flap. In other embodiments, bases (or probes) bind so as to fill in at least a portion of the “gap” left in the strand in which the flap was formed. In these embodiments, the presence of the gap-filling bases or probes serves to fill in the gap such that the flap remains “free” and does not return to its original position. Labeled bases or probes can be bound to the flap and to the gap left behind by the flap's formation.

Suitable labels include fluorescent dye molecules, such as fluoroescein and the like. A non-exhaustive listing of fluorophores is obtainable from Abcam plc, and suitable fluorphores will also be known to those of ordinary skill in the art. Labels may also include magnetic bodies, radioactive bodies, quantum dots, and the like.

When labeled genomic DNA is extended linearly on supporting surfaces or inside nanochannel arrays, the spatial distance between signals from decorated probes hybridized to the sequence specific flaps is quantitatively measurable (in a consistent fashion). This information may then be used to generate unique “barcode” signature patterns that reflect specific genomic sequence information in that region. The nicked gaps on target molecules are suitably created by specific enzymes, including but not limited to Nb.BbvCI; Nb.BsmI; Nb.BsrDI; Nb.BtsI; Nt.AlwI; Nt.BbvCI; Nt.BspQI; Nt.BstNBI; Nt.CviPII and combinations thereof. Based on this map, sequencing can be performed.

As one non-limiting example, a barcode could be formed as follows. A known disease state is characterized by the unique nucleotide sequence TTT-(10 bases)-CCC-(5 bases)-AAA. Three probes are formed: AAA-red dye; GGG-blue dye, and TTT-green dye. The probes are then contacted to a flap-bearing dsDNA sample where the flap has been formed in a region of the dsDNA known to contain the unique nucleotide sequence described above, under conditions that promote probe binding. The DNA sample is then elongated and the user assays the sample for the presence of the probes. If the user detects that the three dyes are present in the sample and are in the appropriate order and are appropriately spaced apart from one another (i.e., the order of dyes is red-blue-green, and the red and blue dyes are separated by a distance that corresponds to 10 bases and the blue and green dyes are separated by a distance that corresponds to about 5 bases), the user will have information that is suggestive that the dsDNA sample in question may possess the known disease.

The above-listed probes are illustrative only. Probes can have a length of 1-10 bases, 1-100 bases, 1-1000 bases, or even larger. Probes may bear a single tag or label or multiple tags or labels. As one example, a probe may be constructed to bear two (or more) fluorophores, or a fluorophore and a radioactive body. A probe can include two or more binding regions (e.g., AAA and CGG) that are connected by a flexible or rigid spacer region.

The claimed invention can also be used to detect copies of a particular sequence or gene. In these embodiments, the user may process DNA to form flaps and contact probes to the DNA, as described elsewhere herein. The presence of two or more “barcodes” that are unique to a particular DNA sequence can then be used to show that an individual may have multiple copies of a particular gene or particular sequence. This can be useful in diagnosing or predicting the presence of a condition that is itself characterized by multiple copies of a gene, such as various polygenic disorders. The user may also use the distance between two or more barcodes (which distance may be determined by elongating the sample) to assist in characterizing a dsDNA sample. For example, the user may use probes to generate barcodes at the beginning and end of a region on a dsDNA sample that is known (or suspected) of containing a region that is critical to expression of a particular disorder.

If the disorder is not present, the distance between the barcodes may be a first distance D0. If, on the other hand, the disorder is present, the distance between the two barcodes may be found to be a longer distance D1. In that case, the user will have information that suggests that the sequence (e.g., gene) of interest is present in the subject that provided the dsDNA sample. In other embodiments, a “normal” individual may possess a gene such that the “normal” distance between the barcodes for the beginning and end of a particular region of DNA is D1. If, however, the individual lacks that gene, the distance between the two barcodes may be the shorter distance D0, in which case the user will have information suggesting that the donor of the dsDNA lacks the base sequence (or gene) of interest.

This information can in turn be used to design a protective (or therapeutic) regimen for the subject or patient. As one example, should the user determine that the subject poses a genetic profile consistent with phenylketonuria, the user can advise the subject to avoid consumption of phenylalanine-containing material.

The present invention is also used to detect the presence of multiple, different base sequences in a dsDNA sample. This may be accomplished by using probes so as to effect different barcodes for different sequences. For example, the user may know that Disease 1 is characterized by base sequences S1a and S1b separated from one another by distance D1. Disease 2 is characterized by base sequences S2a and S2b, separated from one another by distance D2. The user then generates a barcode for Disease 1 (using probes specific or indicative of S1a and S1b) and for Disease 2 (using probes specific or indicative of S2a and S2b). By applying the appropriate probes to a flap-processed dsDNA sample and by interrogating the sample for the presence of the two barcodes, the user can determine whether the donor of the dsDNA sample is characterized as having Disease 1, Disease 2, or both. In this way, the user can assay a single sample for multiple conditions.

The probes used for a particular analysis can be the same or differ from one another in label, binding specificity, or both. For example, a user may perform an analysis using a probe that bears a red fluorescent dye and that binds to the sequence AAA, and a probe that binds to the GTTC sequence, and that bears a green fluorescent dye. The user may use probes that bear magnetic or radioactive bodies simultaneously with probes that bear fluorophores. In this way, the user can assay for multiple probes simultaneously.

The user can also simultaneously assay multiple samples for a single condition. For example, a user can, in parallel, assay multiple dsDNA samples from multiple individuals for a particular condition by assaying those samples for the presence (or lack) of a particular barcode or barcodes. The user can thus also simultaneously assay multiple dsDNA samples for multiple conditions, allowing for high-throughput screening for multiple individuals. In one such embodiment, the user uses a set or array of nanochannels, with each nanochannel being used to elongate processed (e.g., flap-bearing) dsDNA from a different subject. The individual samples are then interrogated (e.g., by application of radiation so as to excite fluorescent probes that may be present on the samples) for the presence of individual probes that indicate the presence of a particular sequence or the presence of barcodes.

The present invention can also be used to generate genetic profiles. In such embodiments, the user may take a dsDNA sample from a subject characterized by a particular condition (e.g., a disease or disorder). The user may then form flaps in the dsDNA at one or more locations and then bind labeled probes to the resultant flaps or gaps in the samples. The user may then interrogate the subject's dsDNA for the presence and location of these probes, which in turn yields information about the content of the subject's dsDNA. (For example, binding of a probe having a sequence ACACAC to the subject's dsDNA indicates that the dsDNA possessed the sequence TGTGTG at that location.)

The user can then construct a map of the subject's DNA, which map is composed of information regarding specific sequences stretches (shown by the binding of probes complementary to those sequences) and the location of those sequences (shown by the location of those bound probes). Thus, the user could, in a non-limiting example, determine that an individual characterized as having genetic disorder X possesses dsDNA having sequence S1 beginning at base location 10,321 of the dsDNA sample and sequence S2 beginning at base location 11,555 of the dsDNA sample.

By treating this information as indicative of the presence of genetic disorder X, the user can then compare dsDNA from another subject against the information from the first subject. If the second subject exhibits sequences S1 and S2 at, respectively, base location 10,321 and 11, 555, the second subject may also likely possess genetic disorder X. In this way, the user can create their own “library” of information regarding the binding locations of various sequence-specific probes onto dsDNA taken from individuals characterized as having various genetic conditions. dsDNA from new subjects can then be processed according to the present invention (e.g., flaps formed and labeled probes then bound) to determine whether the new subjects may have (i.e., carry) one or more disorders that have been cataloged in the user's library of binding information.

In another embodiment, labeled (e.g., covalently tagged) specific sequence motifs of double stranded DNA are created by making nicked single strand gaps, then incorporating labeled nucleotides therein. The physical distribution and frequency of such specific labeled sequence motif along individual unfolded nucleic acid molecules is mapped. In some embodiments, this can be followed by single base sequencing to obtain base-by-base sequence information about the sample.

In another embodiment, individually labeled unfolded nucleic acid molecules are linearly extended. This is accomplished by physically confining such elongated macromolecules within nanoscale channels, topological nanoscale grooves or nanoscale tracks defined by surface properties. As one example, the devices and methods in U.S. patent application Ser. No. 10/484,293 are considered suitable for effecting linear extension. Optical tweezers and shear-stress application methods (e.g., U.S. Pat. No. 6,696,022, incorporated herein by reference) are also considered suitable for effecting such elongation.

In another embodiment, extremely small nanofluidic structures, such as nanochannels, posts, trenches, and the like, are fabricated on a substrate and used as massively parallel arrays for the manipulation and analysis of biomolecules such as DNA and proteins at single molecule resolution. Suitably, the size of the cross sectional area of channels is on the order of the cross sectional area of elongated biomolecules, i.e., on the order of about 1 to about 10⁶ square nanometers, to provide elongated (e.g., characterized as being at least partially linear or partially unfolded) biomolecules that can be individually isolated and analyzed simultaneously by the tens, hundreds, thousands, or even millions.

It is desirable (but not required) that the length of the channels be long enough to accommodate a substantial portion of a macromolecule's length or even a substantial number of macromolecules, ranging from the length of single field of view of a typical CCDA camera with optical magnification (about 100 microns) to as long as an entire chromosome, which can be on the order of 10 centimeters long. The optimal length will depend on the needs of the user.

The present invention also relates to the uses of such labeling processes and features. The flap and single stranded DNA gap can be used in numerous fields including, but not limited in genomics, genetics, clinical diagnostics.

In one embodiment, tagged probes (e.g., with fluorophores) are hybridized on the flaps or single stranded DNA gaps along long double stranded genomic DNA molecules, the labeled DNA molecules can then be imaged under fluorescent microscope to observe spatial barcodes (i.e., signatures related to nucleotide spacing, sequencing, or both) of the labeled flaps or single stranded DNA gaps. The barcodes can in turn be used for whole genome mapping, as signatures from individual barcodes can be pieced together to provide additional information about particular regions of a sample macromolecule. As one non-limiting example, the user may break a DNA sample into subsections and then assay each subsection for the presence (or lack) of particular base sequences and the presence of such sequences in a particular order. After assaying the subsections, the user can assemble information gleaned from individual subsections into an overall information “map” for the entire, original sample.

As one non-liming example, the user may take a 5 kb sample and dissect the sample into 5 1 kb subsections. The user may then form flaps in each of these subsections and assay each subsection for one or more genetic conditions known (or suspected) to be characterized by a base sequence present on that subsection. For example subsection 1 may be assayed for heart disease, where the characteristic sequence or set of sequences is known to occur at positions 0-1000 bases, and subsection 2 may be assayed for diabetes, where the characteristic sequence or set of sequences is known to occur at positions 1001-1999. The user can then assemble this information to arrive at a comprehensive assessment for the disease state of the individual.

In another embodiment, flaps or single stranded DNA of different genomic regions are labeled with differently-colored (or differently-signaled) probes for identifying the relationship of two regions. In one such example, of BCR-ABL fusion, the presence of two colors or more at the same location evidences a structural variation, such as translocation. This is shown in FIGS. 5A-F, which figure illustrates translocation of portions of the BCR and ABL chromosome segments.

In another embodiment, one or more spatial barcoding patterns (which may include patterns that include single colors or multiple colors) of labeled flaps or single stranded DNA gaps can be used to interrogate multiple regions for multiplexed disease diagnostics. As one non-limiting example, the user could interrogate multiple regions for multiple translocations.

This is shown by, e.g., non-limiting FIG. 6. That figure depicts the binding of multiple probes to multiple locations on a DNA sample, enabling the user to assay that sample for the presence of multiple diseases, which assaying can be done simultaneously. As shown in that non-limiting figure, a particular disease (Disease 1) manifested in the BCR-ABL region presents a unique barcode or signature when particular flaps in that region are formed and then labeled by appropriate labels. Disease 2 likewise presents a unique barcode or signature when particular flaps in that region are formed and labeled. A user thus has the capability of assaying for two or more diseases simultaneously, enabling rapid detection of multiple diseases or other states in a given subject. By forming flaps, the user gains an access point into the structure of the DNA sample, which access point can then be used for sequence-specific binding of probes.

The present invention can also be used for performing sequencing of a DNA sample. In such embodiments, the user may form flaps in DNA (providing an access point into the DNA structure). The user can then introduce single-base labeled probes, one at a time, to probe the base-by-base sequence of the DNA sample. For example, the user could introduce a nick in the DNA and then introduce red probe for A. If a red label is then visible, the user will have information that A is present at the nick site. If a red label is not visible, the user can introduce a second labeled probe specific for a different nucleotide.

In another embodiment, the user can also break a DNA sample into fragments, form nicks/flaps along the length of the fragments, and then introduce base- or sequence-specific probes at the nicks/flaps on the fragments. The resulting information gleaned from each fragment can then be assembled back together to develop a sequence map of the original, full-length DNA sample. The nicks/flaps can be formed at specific locations on a DNA sample or at random locations. For example, the user might form a 10-base flap/gap at base position 1 and base position 11 on a 20-base fragment. The user can then introduce various uniquely labeled and uniquely-specific probes (including probes up to 10 bases in length) to the fragment. By determining which probes bound to the fragment (based on the particular signals detected from the bound probes), the user can then obtain sequence information about the fragment.

Probes can be designed to bind to flaps or to single stranded DNA gaps on specific chromosomes. The presence of excess or too few copies of a chromosome can be used for diagnosis of aneuploidy. For example, probes can be designed to label sequences that evidence the presence of a particular gene or even chromosome. The presence of multiple probes (or multiple barcodes related to the presence of the probes) in the subject can then be used to show that the subject possesses multiple copies of the gene or chromosome in question.

In another embodiment, the claimed invention identifies pathogen genomes. The pathogen genomes suitably break into predicted fragments during flap generation, and probes (e.g., so-called universal probes) then used to interrogate the flaps' conserved sequence(s). The barcode pattern thus obtained is then compared to a predicted reference map to enable the user to determine the structure of the genome under analysis. This is known as two layer DNA barcoding, which considers both DNA fragment size and barcodes on each fragments with different size.

In another embodiment, the procedures are used to identify pathogen genomes. The pathogen genomes break into predicted fragments during flap generation, with probes then used to interrogate the flap conserved sequence.

The obtained barcode is then compared to the predicted reference map to yield de novo mapping of the pathogen genome. This is the two layer DNA barcoding scheme, which combines DNA fragment size and barcodes for fragments of different size.

In another embodiment, the procedures identify pathogen genomes. Based on known pathogen genomic sequence, the user may design pathogen specific flap or single stranded DNA gap probes, which result in different barcodes for different pathogens, enabling the user to construct a “library” of the various barcodes indicative of the various pathogens or other sequences of interest. This is shown in non-limiting FIG. 7, which figure demonstrates the application of various, sequence-specific probes to a sample derived from the breast cancer genome to assay for the presence of various segments within that genome.

In another embodiment, flaps or single stranded DNA gaps can be used to enrich specific genomic regions. For example, the hybridization of biotinylated probes to specific region containing specific flap sequences can be effected so as to immobilize the region under analysis. The hybridized DNA molecules are selected by binding to beads or substrates containing avidin molecules. The bound molecules are retained for further genomic analysis, and unbound DNA molecules are washed away. In this way, the user can immobilize DNA for ease of analysis and processing. The flap may be the point of attachment between the sample DNA and the bead or substrate. In other embodiments, the point of binding may be between a base on the main dsDNA and the bead or substrate, as opposed to between a flap and the bead or substrate.

In another embodiment, single base mutation on flap sequences or single stranded DNA gap sequences are obtained for SNP or haplotype information gathering, as shown by non-limiting FIG. 11. In that figure, the A and G alleles of SNP 1 and 2 (respectively) are shown.

### BRIEF DESCRIPTION OF THE DRAWINGS

The summary, as well as the following detailed description, is further understood when read in conjunction with the appended drawings. For the purpose of illustrating the invention, there are shown in the drawings exemplary embodiments of the invention; however, the invention is not limited to the specific methods, compositions, and devices disclosed. In addition, the drawings are not necessarily drawn to scale. In the drawings:

FIG. 1A illustrates a schematic of creating signature “barcoding” pattern on long genomic region with single strand flap generation after nicking. FIG. 1B shows that a sequence-specific nicking endonuclease or nickase creates a single strand cut gap on double stranded DNA, into which a polymerase will bind and begin strand extension while generating displaced strand or so-called “peeled flaps” simultaneously. FIG. 1C shows that these peeled, single stranded flaps create available regions for sequence specific hybridization with labeled probes to generate identifiable signals. Nicking can also be effected by contacting the sample with radiation (e.g., UV radiation), a free radical, or any combination thereof.

FIG. 1D shows labeled genomic DNA being unfolded linearly within a nanochannel array, with the spatial distance between signals from decorated probes hybridized on the sequence specific flaps being measurable and thus generating unique “barcode” signature patterns that reflect a specific genomic sequence present in that region. Multiple nicking sites on a lambda ds-DNA (48.5 kbp total length) are shown as an example created by a specific enzyme, which enzymes include but are not limited to Nb.BbvCI; Nb.BsmI; Nb.BsrDI; Nb.BtsI; Nt.AlwI; Nt.BbvCI; Nt.BspQI; Nt.BstNBI; Nt.CviPII, and any combination of these. A linearized single lambda DNA image showing a fluorescently labeled oligonucleotide probe hybridized to an expected nickase created location is also shown. Such recorded actual barcodes along long biopolymers are designated herein as so-called observed barcodes;

FIGS. 2A-C illustrate the use of lambda DNA molecules as a model system, upon which different labeling schemes are performed. FIG. 2A shows nick-labeling; FIG. 2B shows fluorescent probes having specific sequences hybridized onto two flap structures; and FIG. 2C illustrates signals evolved from labeled nicking sites and labeled flap structures;

FIG. 3 illustrates six base sliding analysis of 50 base pairs of flap sequences across chromosome 22 based on Nb.BbVCI. As shown, a significant conserved sequence was observed on flap sequences. This conserved sequence can in turn be used to design one or more probes to target multiple flap structures;

FIG. 4 illustrates the usage of an exemplary universal probe, TGAGGCAGGAGAAT (SEQ ID NO: 9), which probe was designed to hybridize to 21 flap structures (out of total 52 nicking sites) on a BAC clone 3f5. The barcoding pattern produced therein matched well with the predicted pattern, proving that one can use such universal probes for whole genome mapping;

FIG. 5A-F illustrate clinical diagnosis of translocations for BCR and ABL1 gene translation, which forms the so-called Philadelphia chromosome, the main cause of leukemia. In this scheme, the BCR gene was labeled with green probes at multiple flaps, and the ABL1 gene was labeled with red probes at multiple flaps. If a red and green pattern were observed, the translocation of the two genes was confirmed.

FIG. 6 is a schematic illustration, showing the disclosed method of multiplexed diagnosis. Each disease or gene region forms its own signature barcode, which barcode may include two (or more) colors. Placing multiple barcodes on multiple flaps provides the user with an essentially unlimited barcoding capability;

FIG. 7 depicts the validation of a structural variation, in which a BAC clone 3f5 having multiple structural rearrangements was confirmed by flap mapping;

FIG. 8 is a schematic illustration of pathogen identification using universal probes with two layer barcodes, fragment size and flap barcoding;

FIG. 9 illustrates pathogen identification using pathogen specific probes; the probes are designed to target specific region or regions of the pathogen genome, which labeled structure forms a unique barcode. In this case, 350000-400000 and 1090000-1130000 of Salmonella regions were used as the examples; a region of E. coli is also shown;

FIG. 10 is a schematic illustration of sample enrichment and diagnosis; and

FIG. 11 illustrates molecular haplotyping based on flap structures.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, reference to values stated in ranges include each and every value within that range.

In a first embodiment, the present invention provides methods of obtaining structural information from a DNA or other nucleic acid sample. These methods suitably include processing a double-stranded DNA sample so as to give rise to a flap of the first strand of the double-stranded DNA sample being displaced from the double-stranded DNA sample. The flap suitably has a length in the range of from about 1 to about 1000 bases, or from 5 to 750 bases, or from 10 to 200 bases, or from 50 to 100 bases. The optimal length of the flap will depend on the needs of the user. As explained elsewhere herein, the formation of the flap results in a “gap” being formed in the dsDNA opposite the flap.

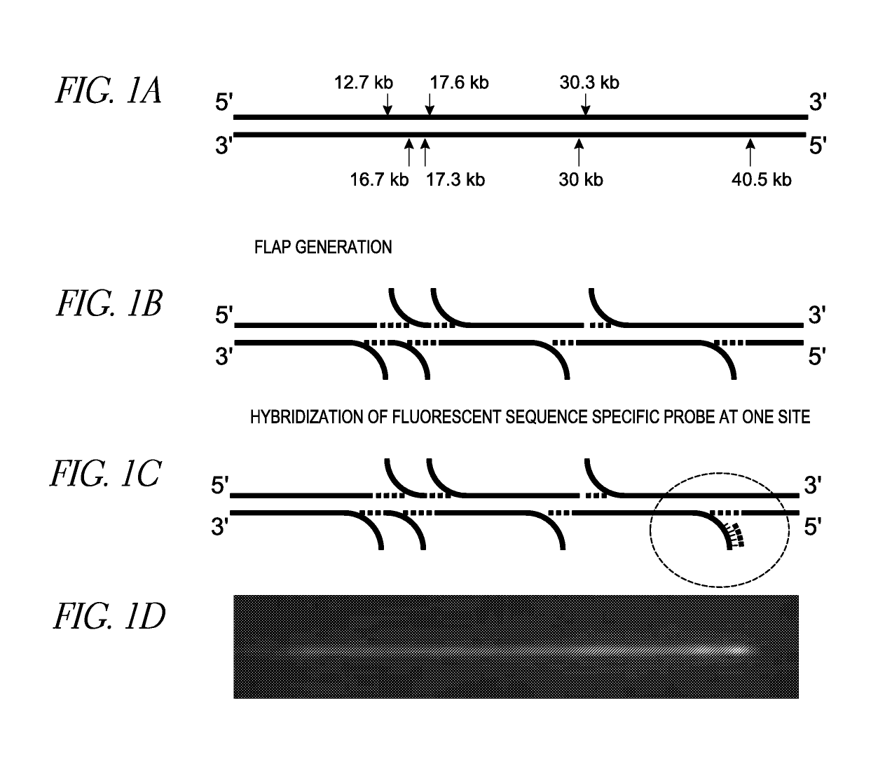

Creation of the flap suitably gives rise to a gap in dsDNA sample that corresponds to the flap location, as shown by, e.g., FIG. 1. This flap (and gap) can thus be used to expose a single-stranded portion of the dsDNA for amplification, probing, or further labeling. Thus, the user may perform genetic analysis of DNA or other nucleic acid biopolymer samples without having to break the biopolymer into individual nucleic acids for analysis. Moreover, the present invention enables the user to perform an analysis of a nucleic acid biopolymer that can be essentially independent of the sequence of the nucleic acids within the biopolymer.

This is so because genetic information can be gleaned from the mere size/length of a DNA region that is flanked by two or more probes. For example, if probes are bound to a sample so as to flank a region of interest and it is seen that the region of interest is longer than is normally seen (or longer than should be seen) in a subject, the user will know that the subject may be disposed to a physiological condition or disease characterized by a lengthened region of interest, such as a condition characterized by excessive copy numbers of a particular gene.

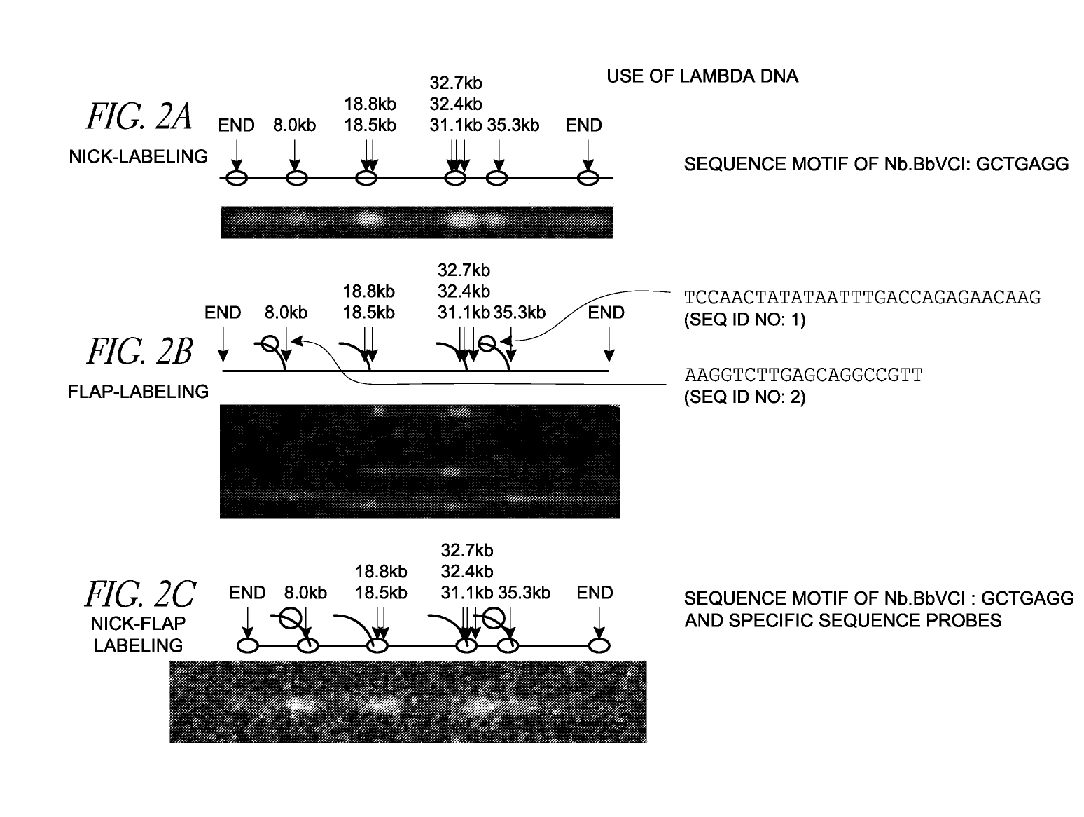

One or more replacement bases is suitably incorporated into the first strand of double-stranded DNA so as to eliminate the gap, and at least a portion of the double-stranded sample thus evolved is suitably labeled with one or more tags. Tags are suitably fluorescent labels, radioactive labels, and the like. Labels may be disposed (see, e.g., FIG. 2) at nicks or flaps along the length of a macromolecule, or at any combination of these locations. Labels (e.g., borne by probes) may be introduced into the gap of the dsDNA, as well.

Nicking is suitably effected at one or more sequence-specific locations. This may be accomplished by, e.g, a nickase or a nicking endonucleoase, or by any enzyme introducing a single stranded break, by an electromagnetic wave (e.g., ultraviolet light), by free radicals, and the like. The nicking may also be accomplished at a non-sequence-specific location. Enzymes for creating such flaps are commercially available, e.g., from New England Biolabs.

Incorporation of the aforementioned replacement bases may be accomplished by contacting the first strand of double-stranded DNA with a polymerase, one or more nucleotides, a ligase, or any combination thereof. This is, in some embodiments, performed in the presence of one or more replacement bases, which bases may include tags or labels that are detectable. In this way, the user may incorporate into a target labels or tags that in turn allow the user to obtain structural information about the target macromolecule.

The generation of flap structure is suitably controlled by polymerase extension and incorporation of one or more nucleotides, as is known in the art. The polymerase suitably possessed 5′-3′ displacement activity and, in some embodiments, lacks 5′-3′ exonuclease activity. Suitable polymerases include—but are not limited to—vent exo-polymerase (New England Biolabs).

The polymerase and the nucleotides may be chosen so as to control the length of the flap. Reaction temperature and time can also be modulated so as to control the length of the flap evolved. Flap length may also be controlled by the relative proportions of the different nucleotides present, i.e., the ratio of dATP, dCTP, dTTP, and dGTP. The ratio of the nucleotides to polymer terminator can also affect flap length; terminators can include (but are not limited to) to ddNTP, and acylo-dNTP.

Labeling is suitably accomplished by (a) binding at least one complementary probe to at least a portion of the flap, the probe suitably comprising one or more tags (e.g., fluorophores), by (b) two or more complementary probes hybridized next to each other and can be ligated together, or even by (c) two or more complementary probes hybridized next to each other with a gap of one or more bases between them. The gap can then be filled with labeled or non labeled nucleotides, which nucleotides can be connected by way of a ligase. Labels may be present on flaps, into the resultant “gap,” or in multiple locations.

Also provided are methods of obtaining structural information from a DNA sample. These methods include processing a double-stranded DNA sample so as to give rise to a single stranded DNA gap of the second strand of the double-stranded DNA sample. This may be accomplished by, e.g., the first strand DNA being digested at the nicking site from the dsDNA DNA sample. The gap suitably has a length in the range of from about 1 to about 1000 bases, or from 5 to 750 bases, or even from 100 to 500 bases. The user suitably labels at least a portion of the single stranded DNA gap.

Nicking is accomplished by nicking a first strand of double stranded DNA molecules, as described elsewhere herein. The nicking endonuclease Nb.BbvCI is considered suitable. Other suitable nicking endonucleases are available from commercial sources, including New England Biolabs, and Fermentas.

In some embodiments, the strand downstream from the nick is extended, e.g., with dUTP dA(C,G)TP, by a 5′>3′ exo+ polymerase. Vent polymerase is one such suitable enzyme for this.

The DNA is then digested, e.g., with a uracil DNA glycosylase. The removal of the dUTP generates the single stranded DNA gap.

In some embodiments, the flap can be removed in part or in its entirety. The resultant gap is then filled in with a flap endonuclease, which gives rise to a single stranded DNA gap structure. The extended sequence will be nicked again with the same nicking endonuclease and the sequence will be removed by denaturing.

Labeling is suitably accomplished by (a) binding at least one complementary probe to at least a portion of the flap, the probe comprising one or more tags, by (b) two or more complementary probes hybridized next to each other and can be ligated together, and/or by (c) two or more complementary probes hybridized next to each other with one or more base gap between them. The gap (or gaps) can then be filled with labeled or non labeled nucleotides and ligated together with ligase.

The labeled samples may then be elongated, as described elsewhere herein. The elongation may be accomplished by entropic confinement, by application of flow or shear forces, by optical tweezers, by application of magnetic forces (e.g., where the sample includes a magnetic material, such as a bead), and the like.

Methods of obtaining structural information from DNA are also provided. These methods include labeling, on a first double-stranded DNA sample, one or more sequence-specific locations on the first sample; labeling, on a second double-stranded DNA sample, the corresponding one or more sequence-specific locations on the second double-stranded DNA sample; elongating at least a portion of the first double-stranded DNA sample; elongating at least a portion of the first double-stranded DNA sample; and comparing the intensity, location, or both of a signal of the at least one label of the first, elongated double-stranded DNA sample to the intensity of the signal of the at least one label of the second, elongated double-stranded DNA sample.

In this aspect of the invention, the user compares the barcode or probe-binding profiles of two (or more) samples. This enables the user to compare the genetic profile between a sample from an individual known to have (or lack) a particular condition with a sample from a second individual, enabling the determination of the disease state of the second individual. For example, a user may compare the probe profiles of an individual known to be positive for a disease that can be detected by genome analysis (e.g., diabetes) and the profile of a test individual who has not been tested for that disease. If the two profiles are identical (e.g., if the test individual exhibits the same “barcodes” as the positive control individual), the user will have information that is suggestive of the test individual being “positive” for the disease.

As described elsewhere herein, this is suitably accomplished by hybridizing one or more probes to at least one of the DNA samples. This may be accomplished by the flap-based methods described elsewhere herein.

As described elsewhere herein, labeling is accomplished by nicking a first strand of a double-stranded DNA sample so as to give rise to (a) flap of the first strand being separated from the double-stranded DNA sample, and (b) a gap in the first strand of the double-stranded DNA sample corresponding to the flap, the gap defined by the site of the nicking and the site of the flap's junction with the first strand of the double-stranded DNA sample.

The methods suitably use probes that are designed for whole genome mapping, which probes conserved flap sequences across the whole genome. In this way, one or only a few probes can hybridize to hundred or tens of thousands of flap sequences, taking advantage of the sequence or sequences that are conserved across these flaps. The hybridized probes suitably form a barcode to identify each individual DNA fragment, where the barcode is unique to a particular fragment. Probes can be sequence-specific.

A variety of schemes can be used for genome mapping. In one embodiment, nick labeling plus flap labeling (two or more colors) can be used. In another embodiment, one nicking enzyme and flap labeling with two or more probes with two or more different colors can be used. In yet another embodiment, two different nicking enzymes with various combination of flap and nick-labeling can be used.

Other methods for obtaining structural information from DNA are also provided. These methods include labeling different (e.g., two or more) regions of a flap with differently-colored probes so as to identify the spatial relationship between the two regions. Alternatively, the user may label the flaps of different regions with different color probes and different numbers of probes for identifying the relationship of two regions. Users may also label flaps of different regions with different numbers of differently (or similarly) colored probes and use the resultant color patterns to identify the spatial relationship between two or more regions. Labeling may be effected on flaps of different regions with different probes. The probes may also be targeted to particular chromosomes for identifying specific chromosomes.

Probes can be deployed so as to screen for the presence of a single disease or abnormality. Probes can also be used in a multiplexed fashion so as to identify multiple regions and even multiple diseases at the same time. In such embodiments, the user may

Pathogenic genomic material may be identified by probing the flaps or ssDNA gaps. This identification suitably includes using universal probes that bind to sequences conserved across multiple regions, and the universal probes can be used de novo pathogen identification. In one embodiment, this is accomplished by the pathogen genome breaking into predicted fragments during flap generation, with the universal probes being used to interrogate the flap conserved sequence. The obtained barcodes are then compared to the predicted reference map of the pathogen genome. This is known as “two-layer” DNA barcoding, which combines DNA fragment size and barcode information.

FIG. 8 illustrates one example of this two-layered barcoding. As shown in that figure, universal (or other) probes are bound to a sample macromolecule at flap, nick, or both locations. The macromolecule can be subdivided into fragments of certain sizes, and the sizes of the fragments can be used to glean further structural information about the sample. As one non-limiting example, the user—knowing the locations on the original sample that define the endpoints of a given fragment or fragments—can correlate the size of a particular fragment to the location of that fragment within the original sample.

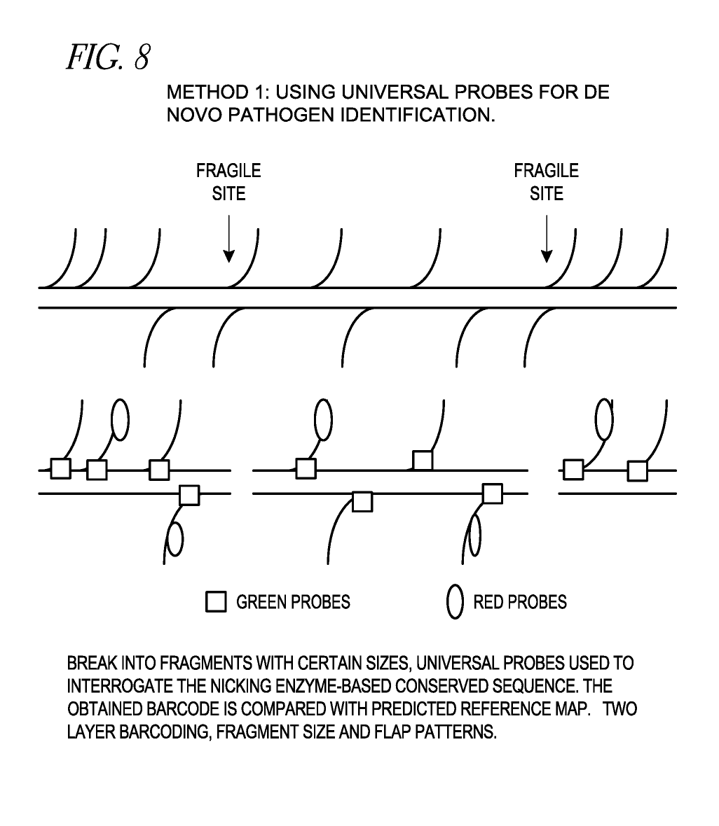

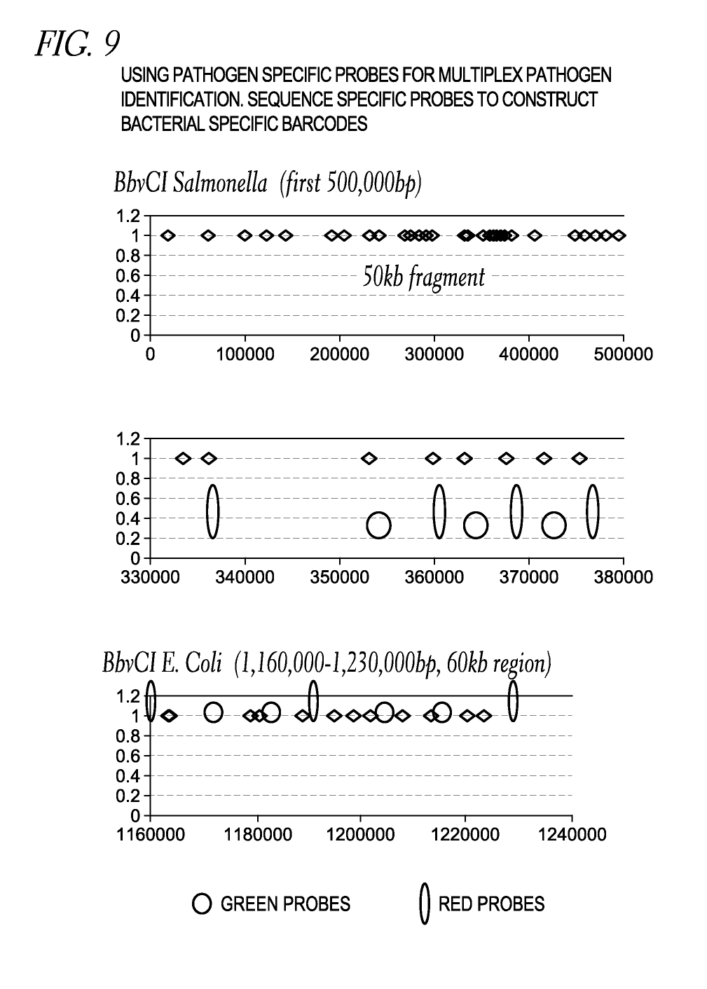

Also provided is the use of pathogen-specific probes for multiplexed pathogen identification. This is accomplished by using a known pathogen genomic sequence to design pathogen-specific flap probes, with different pathogens having different barcodes. As shown in non-limiting FIG. 9, the presence of green-red-green-red probes in that order signifies the presence of Salmonella. The same barcode can be assayed in other regions of the same bacteria. This aspect of the present invention enables the user to use sequence-specific probes that are in turn used to generate pathogen-specific (e.g., bacteria) barcodes.

Such barcodes can then be used to assay for the presence of the pathogen (or even a portion of the pathogen's genome) in a particular sample. As described herein, the user may determine the position of one or more probes based on a signal unique to the region upon which the one or more probes reside; and compare the position, color, or both of one or more probes bound to the DNA sample to a corresponding signal from a DNA region known to correspond to one or more pathogenic states. In this way, the user can determine whether a subject is suffering (or is inclined to suffer) from the pathogenic state.

In another aspect, the present invention provides methods of enriching certain genomic regions. These methods include hybridization of anchor-bearing probes to one or more regions that contain specific flap sequences. (One suitable such probe is a biotinylated probe.) The hybridized DNA molecules can be bound to, e.g., beads or glass surfaces that bear linker molecules, such as avidin. The unbound DNA molecules are washed away, and the bound molecules are then available for further analysis, imaging, and the like. In another embodiment, magnetic beads may be bound or affixed to the DNA sample, and the sample then magnetized to a substrate so as to immobilize the sample.

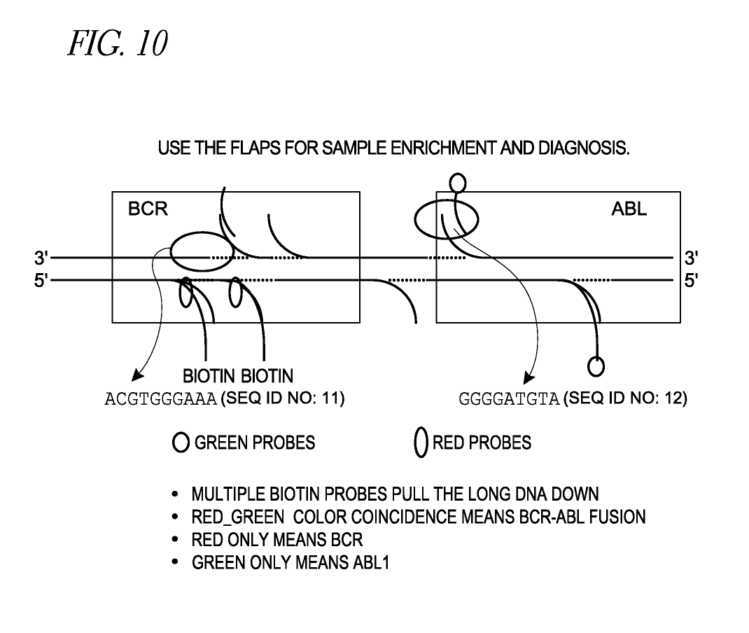

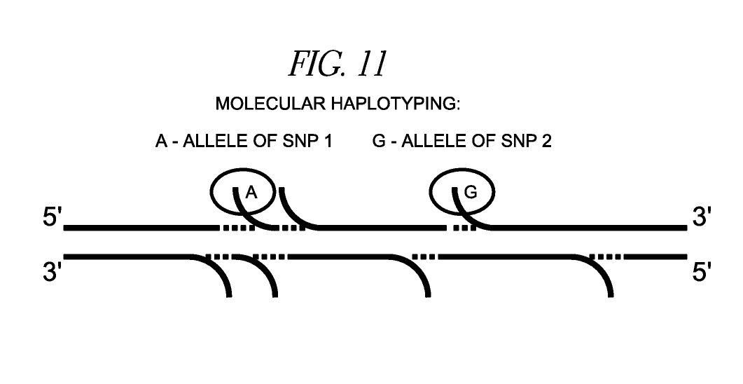

FIG. 10 is a sample, non-limiting embodiment of the inventive techniques. As shown in that figure, probes may be bound to the flaps formed on a DNA sample, as well as inserted into the gap left behind by the formation of the flap. Biotinylated probes secure the flaps to a substrate. In the example shown in that figure, the appearance of both red and green probes signifies the presence of BCR-ABL fusion. If only green probes are shown, only ABL is visible. If only red probes are shown, only BCR is present. Molecular haplotyping can also be accomplished by interrogating single base mutations on flap sequences and single stranded DNA gap sequences.

Also provided are systems suitable for sorting and linearly unfolding such labeled macromolecules in massive parallel fashion for optical and non-optical signal analysis. These systems include, in exemplary embodiments, one or more reaction zones where DNA, RNA, or other sample material undergoes nicking, flap formation, labeling, and the other steps described herein. Such sites may be a reaction vessel—such as a tube, a flask, or other commonly-available laboratory items. Alternatively, one or more of these steps may be performed in a reaction zone in fluid communication with a nanochannel or nanochannel array that is then used to—as described elsewhere herein—elongate the macromolecule so as to allow the user to gather structural information about the macromolecule. The elongation may be accomplished by physical/entropic confinement, by shear fluid flow, by physical force (optical tweezers), and the like. Suitable nanochannel chips and arrays are described in U.S. application Ser. No. 10/484,293, the entirety of which is incorporated herein by reference.

The systems may also include a device—such as an imager—to gather visual information about a labeled sample. In one embodiment, the imager comprises one or more sources of radiation (e.g., light, lasers, and the like) used to excite labels that may be present on macromolecules processed according to the claimed invention. The imager suitably includes a CCD device or other image-gathering hardware. The images may be inspected by the user or be processed and further analyzed by the system. Such further processing may include refinement of the raw image obtained from the labeled macromolecule, as well as comparison of the image obtained from the labeled macromolecule with a model or predicted image generated by analysis of other sample materials or of material that is comparative to the sample being analyzed. The comparison may be performed between an image taken from the nucleic acid biopolymer under analysis and a control image that represents a disease state, a healthy state, or other genetic variation. The comparison may be accomplished (or aided) by a computer.

Additional Disclosure

This application presents methods relating to DNA mapping and sequencing, including methods for making long genomic DNA, methods of sequence specific tagging and a DNA barcoding strategy based on direct imaging of individual DNA molecules and localization of multiple sequence motifs or polymorphic sites on a single DNA molecule inside the nanochannel (<500 nm in diameter, in suitable embodiments). These methods obtain continuous base by base sequencing information, within the context of the DNA map.

Compared with prior methods, the disclosed method of DNA mapping provides improved labeling efficiency, more stable labeling, high sensitivity and better resolution; the disclosed method of DNA sequencing provide base reads in the long template context, easy to assemble and information not available from other sequencing technologies, such as haplotpye, and structural variations.

In a DNA mapping application, individual genomic DNA molecules or long-range PCR fragments were labeled with fluorescent dyes at specific sequence motifs. The labeled DNA molecules were then stretched into linear form inside nanochannel and imaged using fluorescence microscopy. By determining the positions and colors of the fluorescent labels with respect to the DNA backbone, the distribution of the sequence motifs can be established with accuracy, in a manner similar to reading a barcode. This DNA barcoding method is applied, e.g., in the identification of lambda phage DNA molecules and human bac-clones.

One sample embodiment with flap sequences at sequence specific nicking sites comprises the steps of:

a) nicking one strand of a long (e.g., >2 Kb) double stranded genomic DNA molecule with a nicking endonucleases to introduce nicks at specific sequence motifs;

b) incorporating fluorescent dye-labeled nucleotides or none fluorescent dye-labeled nucleotides at the nicks with a DNA polymerase, displacing the downstream strand to generate flap sequences;

c) labeling the flap sequences by polymerase incorporation of labeled nucleotides; or by direct hybridization of the fluorescent probes; or by ligation of the fluorescent probes with ligases.

d) elongating the labeled DNA molecule into linear form within nanochannels by flowing the sample through the channels or by fixing one end of the DNA inside the channels; and

e) determining the positions of the fluorescent labels with respect to the DNA backbone using fluorescence microscopy to obtain a map or signature barcode of the DNA.

Another embodiment having a ssDNA gap at sequence specific nicking sites includes the steps of:

a) nicking one strand of a long (e.g., >2 Kb) double stranded genomic DNA molecule with a nicking endonucleases to introduce nicks at specific sequence motifs;

b) incorporating fluorescent dye-labeled nucleotides or non-fluorescent dye-labeled nucleotides at the nicks via a DNA polymerase, displacing the downstream strand to generate flap sequences;

c) employing the same nicking endonuclease to nick newly extended strand and cutting the newly formed flap sequences with flap endonucleases (detached ssDNA can be removed by increasing the temperature).

d) labeling the ssDNA gap by polymerase incorporation of labeled nucleotides; or direct hybridization of the fluorescent probes; or ligation of the fluorescent probes with ligases;

e) elongating the labeled DNA molecule into linear form inside nano-channels either flowing through the channels or fixed one end of the DNA inside the channels; and

f) determining the positions of the fluorescent labels with respect to the DNA backbone using fluorescence microscopy to obtain a map or barcode of the DNA.

Another application of flaps and single stranded DNA gaps is whole genome mapping. Flaps and/or ssDNA gap sequences of whole genomic DNA made by a nicking endonuclease (including but not limited to Nb.BbVCI), were analyzed and the hybridization probes were designed based on sequences conserved (i.e., present) across multiple regions of a sample or across multiple samples. A single or a few (less than 4 probes) can be used, such as cy3-TGAGGCAGGAGAAT-cy3 (SEQ ID NO: 4). The labeled DNA molecules are linearized in nanochannels (as described elsewhere herein) and DNA barcodes are generated.

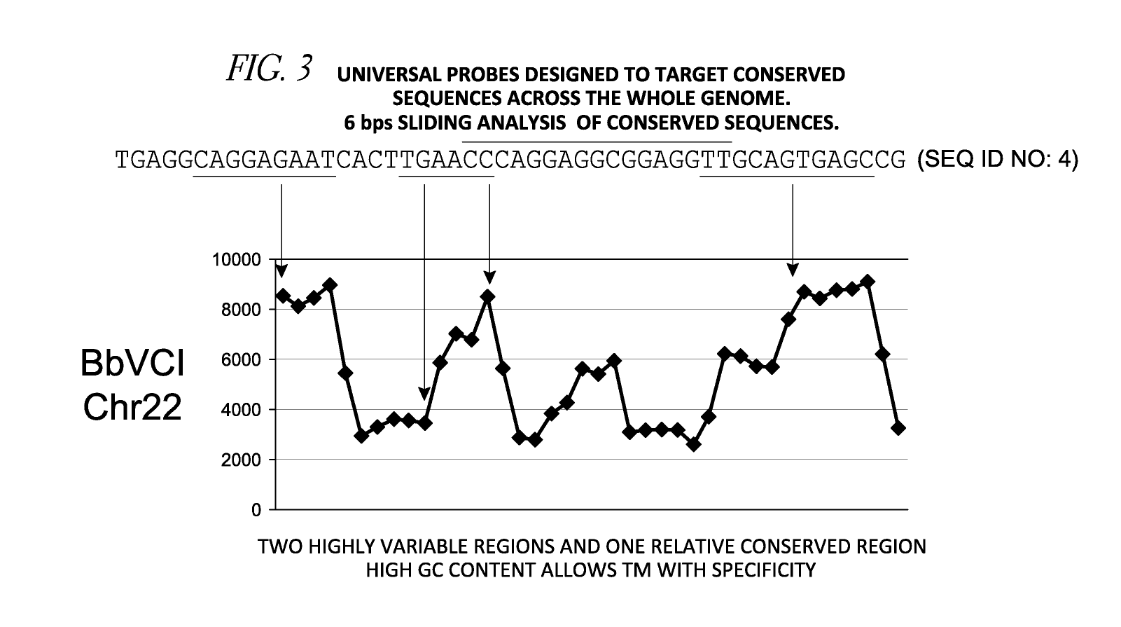

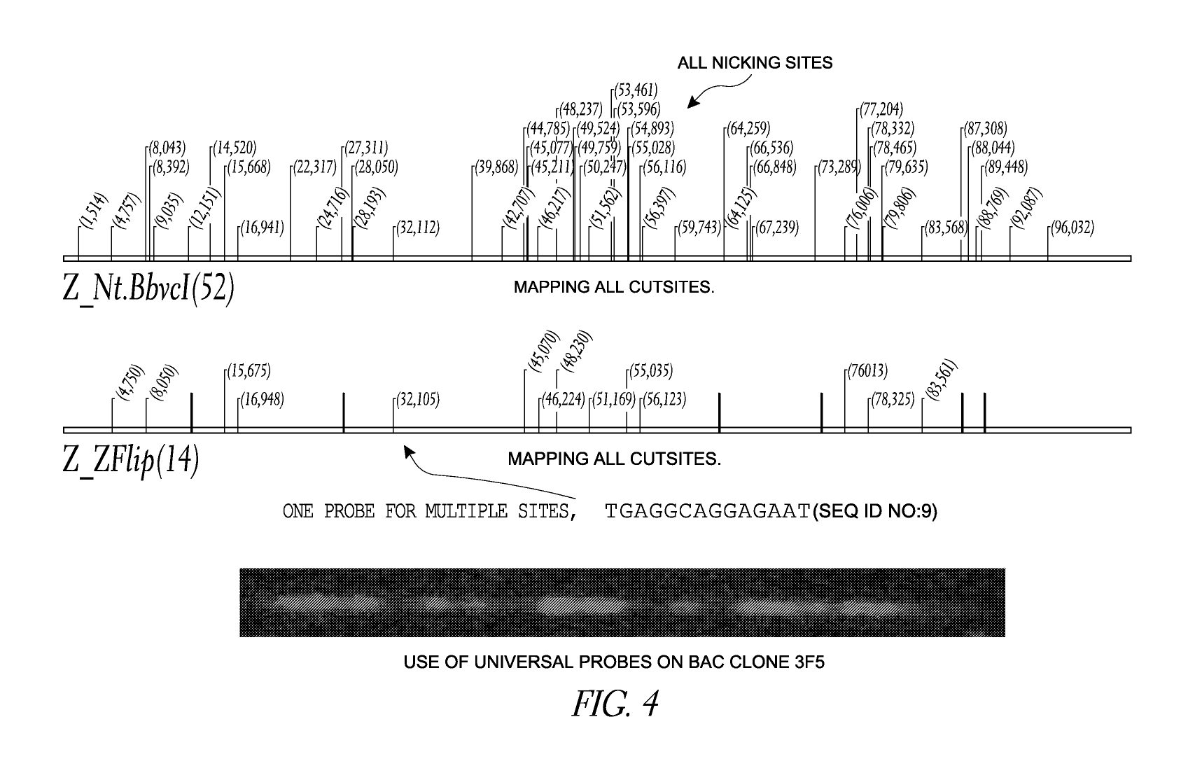

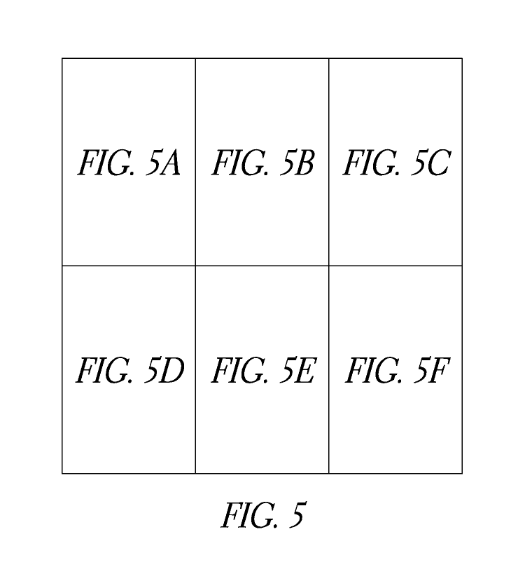

FIG. 3 is an exemplary embodiment showing the use of so-called universal probes to bind and locate conserved regions. As shown in that figure, probes (in this case, a probe that happens to have a comparatively high GC content) can be used to target and locate conserved sequences along the length of a given sample macromolecule. The use of universal probes is further illustrated in FIG. 4, which figure illustrates the use of a single, universal probe that binds to multiple sites along the length of a sample macromolecule.

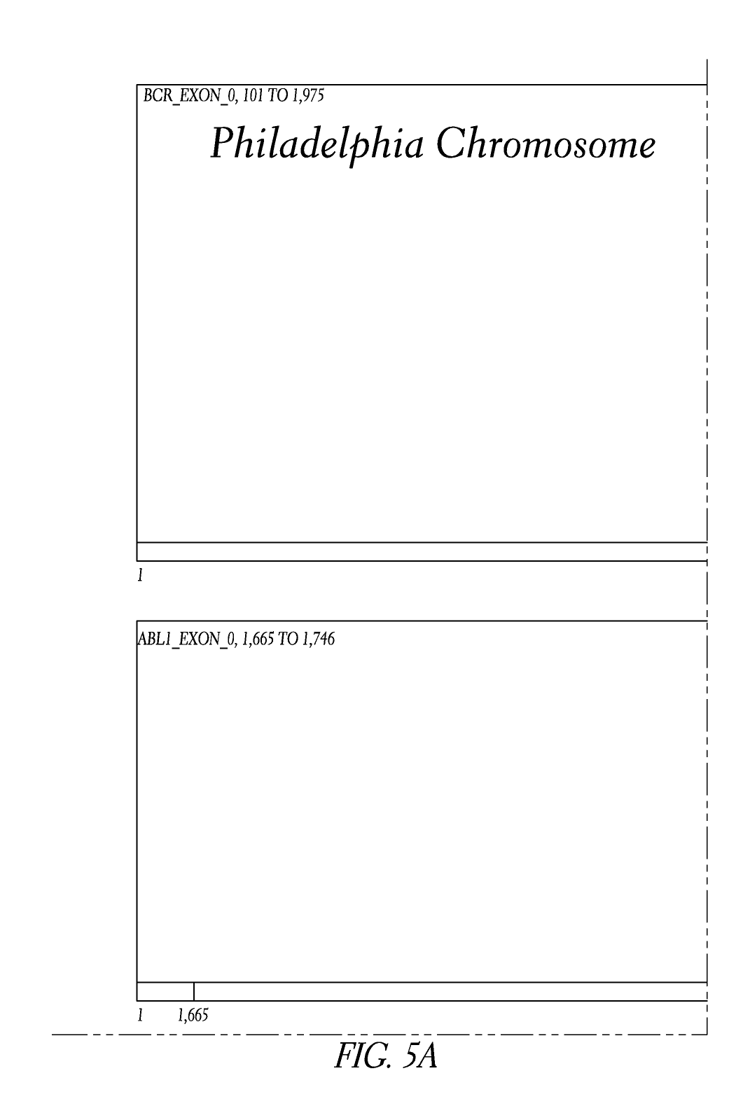

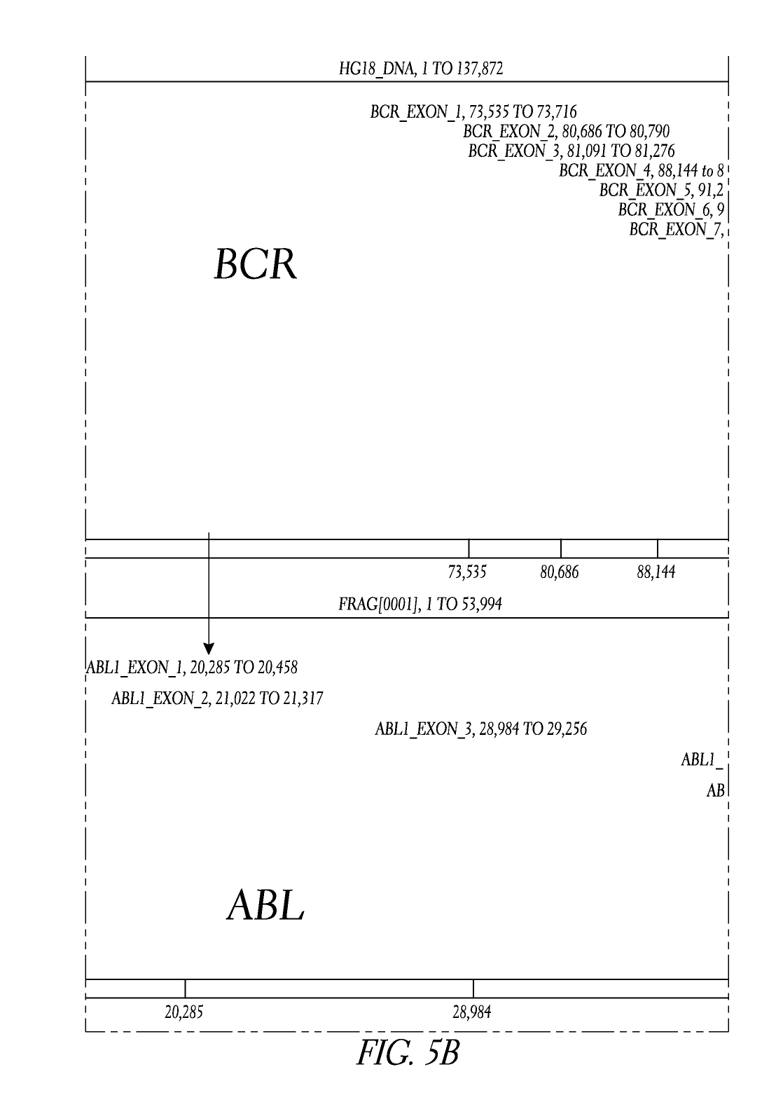

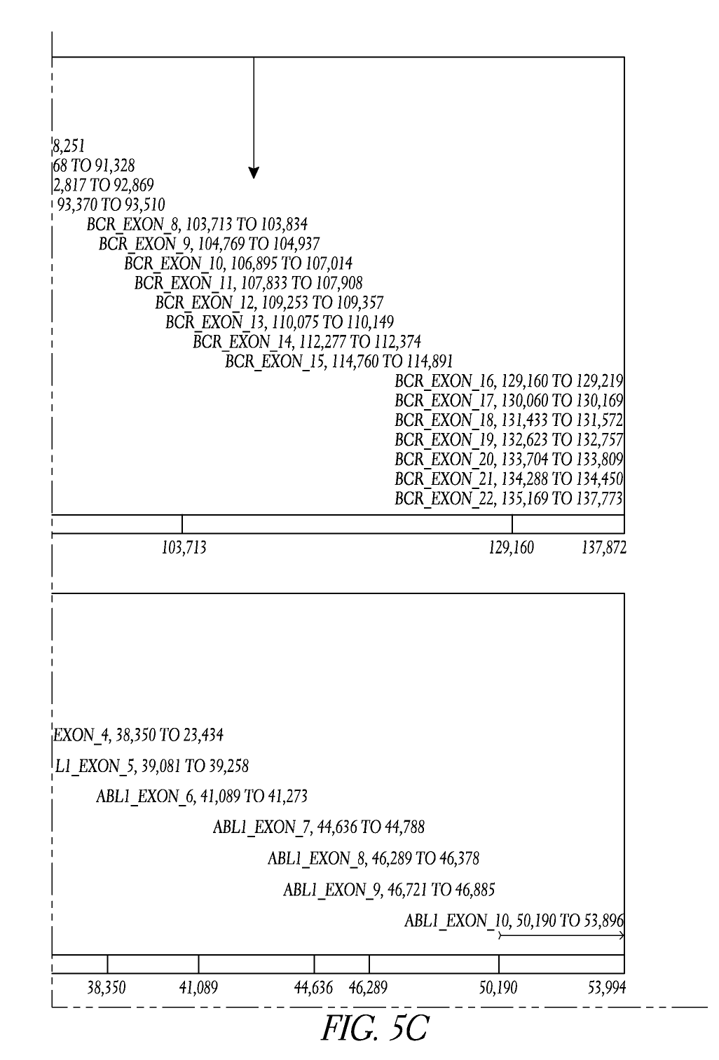

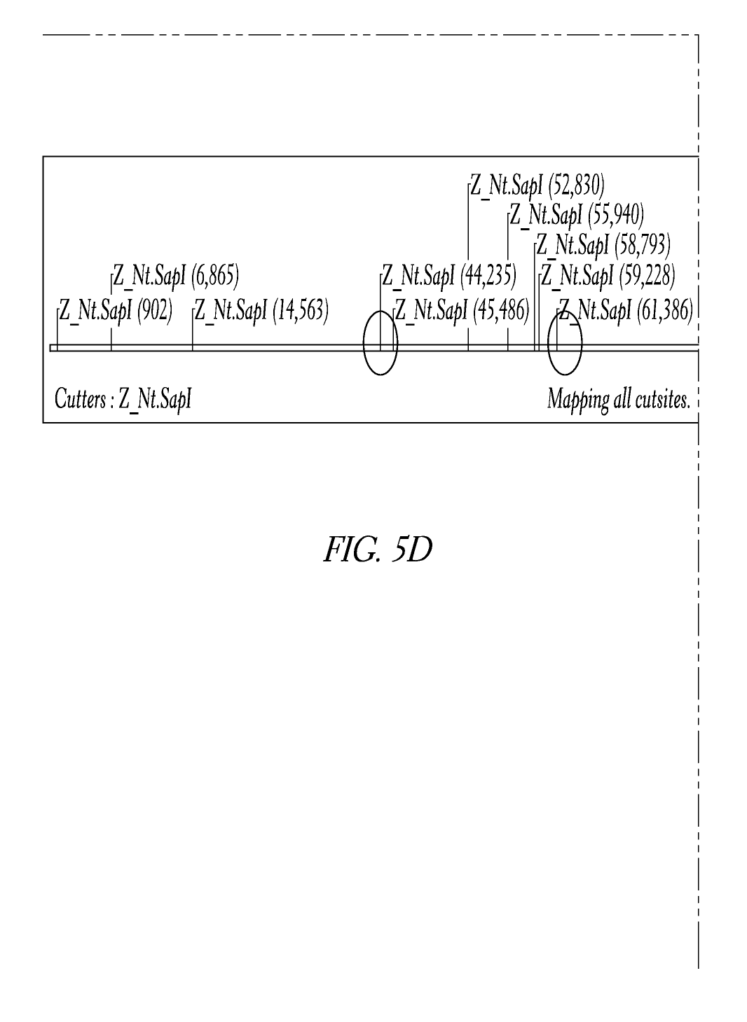

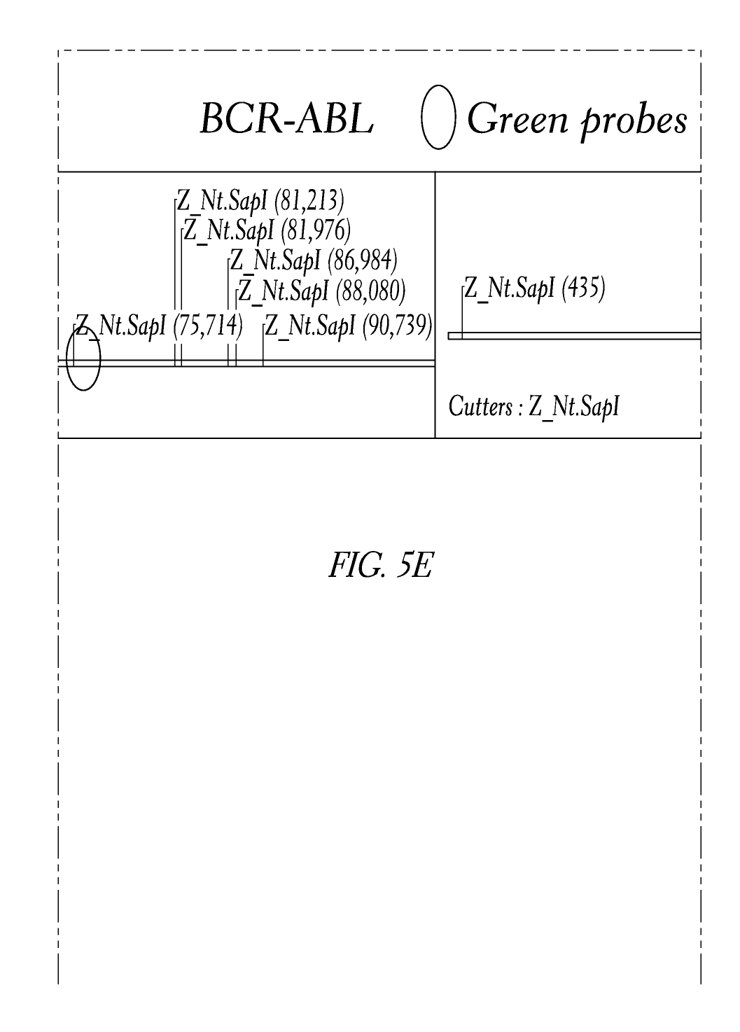

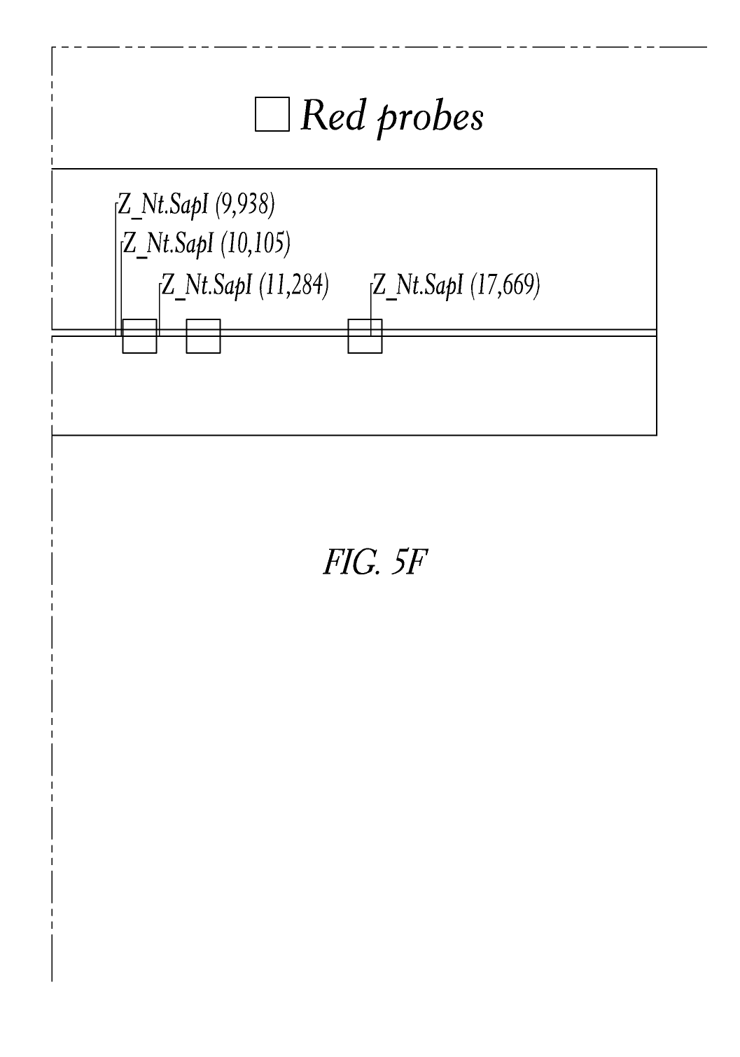

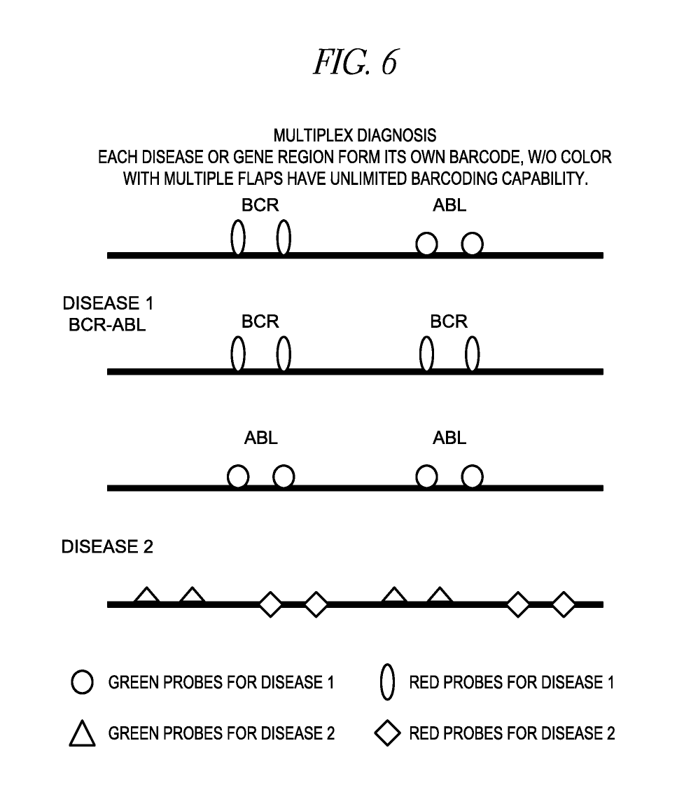

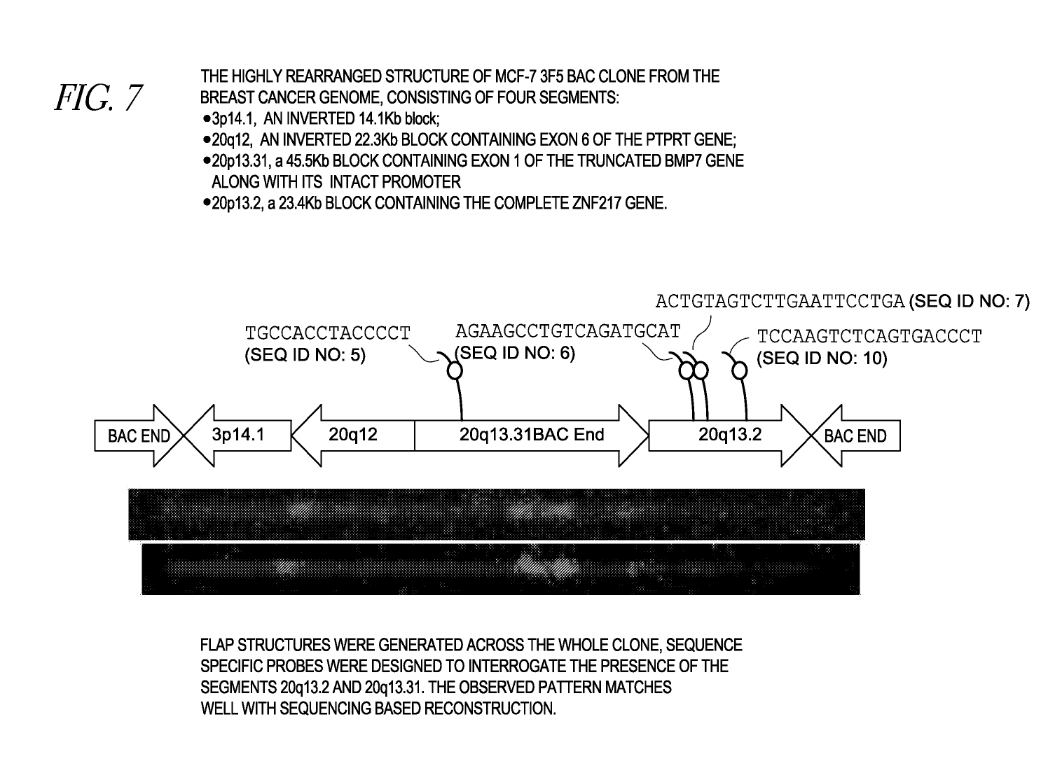

Another embodiment of using the flaps and/or ssDNA gaps is the detection of diseases caused by structural variations. One example of such a disease is BCR ABL gene fusion, which condition is a main cause of leukemia. In this case (as shown by FIGS. 5 and 6), green fluorophore tagged probes hybridize on the flaps or to single stranded DNA gaps of BCR gene, and red fluorophore tagged probes will hybridize on the flaps or to single stranded DNA gaps of the ABL gene. If two color green-red are observed on the same DNA molecules, the presence of BCR-ABL fusion gene is confirmed.

Another embodiment of above diseases diagnosis involves more than two region rearrangements, such as Zinc Finger Breast Cancer Diagnostic Markers, which comprise a 4 segment rearrangement from 4 different regions of genome.

In another embodiment, two or more diseases can be tested either with more color combinations or with more complex flap or ssDNA gap spatial barcodes or both color and the spatial distribution of color flaps and ssDNA gaps a multiplex detection format.

In another embodiment, the procedures are used to identify pathogen genomes. The genomes are suitably nicked at a first strand of double stranded DNA molecules with a nicking endonuclease (including but not limited to Nb.BbVCI, Nb.BsmI, and the like). The two nicking sites suitably sit on opposite strands within 100 bp, which strands suitably break due to flap generation. The breakage pattern will be specific to the specific pathogen genome, which pattern can be used as a first layer of barcode information.

Each subset of the fragments can then be labeled with fluorescent probes on the flaps or ssDNA gaps use a universal probe. The combination of the fragment size and the internal color barcodes then identifies the pathogen genomes. For example, Yersinia bacteria can be indentified in this fashion.

In another embodiment, based on known pathogen genomic sequence, one can choose a particular region of the pathogen genome to confirm the presence of the pathogen. In this case, pathogen specific flap or single stranded DNA gap probes can be designed, which results in specific patterns for different pathogens. For example, Salmonella bacterial genome at the 350000-400000 bp location (a 50 kb region) can be nick-flap labeled with Nb.BbVCI and associated probes to barcode the genome. To increase the specificity, additional such regions can be used, such as a 50 kb region from 1,000,000-1,500,000 bp. Mixtures of pathogen genomes can be identified in a similar fashion.

In another embodiment, the flap or single stranded DNA gaps can be used for the enrichment of specific genomic regions. In these embodiments, the user effects hybridization of biotinylated probes to specific region containing specific flap sequences. The hybridized DNA molecules are then selected by binding them to beads or glass surface containing avidin molecules. The bound molecules are retained for further genomic analysis. The unbound DNA molecules are washed away, and the immobilized samples are subjected to further analysis.

### EXAMPLES

The following examples are illustrative only and do not necessarily limit the scope of the claimed invention.

### Example

### Generating Single Stranded DNA Flaps on Double Stranded DNA Molecules

Genomic DNA samples were diluted to 50 ng for use in the nicking reaction. 10 uL of Lambda DNA (50 ng/uL) were added to a 0.2 mL PCR centrifuge tube followed by 2 uL of 10× NE Buffer #2 and 3 uL of nicking endonucleases, including but not limited to Nb.BbvCI; Nb.BsmI; Nb.BsrDI; Nb.BtsI; Nt.AlwI; Nt.BbvCI; Nt.BspQI; Nt.BstNBI; Nt.CviPII. The mixture was incubated at 37 degrees for one hour.

After the nicking reaction completes, the experiment proceeded with limited polymerase extension at the nicking sites to displace the 3′ down stream strand and form a single stranded flap. The flap generation reaction mix consisted of 15 μl of nicking product and 5 μl of incorporation mix containing 2 μl of 10× buffer, 0.5 μl of polymerase including (but not limited to) vent(exon-), Bst and Phi29 polymerase and 1 μl nucleotides at various concentration from 1 uM to 1 mM. The flap generation reaction mixture was incubated at 55 degrees. The length of the flap was controlled by the incubation time, the polymerases employed and the amount of nucleotides used.

### Example

### Fluorescently Labeling Sequence Specific Nicks on Double Stranded DNA Molecules

Genomic DNA samples were diluted to 50 ng for use in the nicking reaction. 10 uL of Lambda DNA (50 ng/uL) were added to a 0.2 mL PCR centrifuge tube followed by 2 uL of 10× NE Buffer #2 and 3 uL of nicking endonucleases, including but not limited to Nb.BbvCI; Nb.BsmI; Nb.BsrDI; Nb.BtsI; Nt.AlwI; Nt.BbvCI; Nt.BspQI; Nt.BstNBI; and Nt.CviPII. The mixture was incubated at 37 degrees for one hour.

After the nicking reaction completes, the experiment proceeded with polymerase extension to incorporate dye nucleotides onto the nicking sites. In one embodiment, a single fluorescent nucleotide terminator was incorporated. In another embodiment, multiple fluorescent nucleotides were incorporated. The incorporation mix consisted of 15 μl of nicking product and 5 μl of incorporation mix containing 2 μl of 10× buffer, 0.5 μl of polymerase including but not limited to vent(exon-), 1 μl fluorescent dye nucleotides or nucleotide terminators including (but not limited to) cy3, alexa labeled nucleotides. The incorporation mixture was incubated at 55 degrees for 30 minutes.

### Example

### Two-Color Labeling of Nicking Sites and Single Stranded DNA Flaps on Double Stranded DNA Molecules

The nicking sites were labeled with one color fluorophore. The reaction was chased with 250 nM unlabeled nucleotide dNTP to generate flaps. Once the flap sequence were generated, the flaps are labeled with different color fluorescent dye molecules. This is accomplished by, e.g., hybridization of probe, incorporation of fluorescent nucleotide with polymerase and ligation of fluorescent probes.

### Example

### Whole Genome Mapping with a Single Probe TGAGGCAGGAGAAT (SEQ ID NO: 9)

Genomic DNA samples were diluted to 50 ng for use in the nicking reaction. Genomic DNA samples were diluted to 50 ng for use in the nicking reaction. 10 uL of Lambda DNA (50 ng/uL) were added to a 0.2 mL PCR centrifuge tube followed by 2 uL of 10× NE Buffer #2 and 3 uL of nicking endonucleases, including but not limited to Nb.BbvCI; Nb.BsmI; Nb.BsrDI; Nb.BtsI; Nt.AlwI; Nt.BbvCI; Nt.BspQI; Nt.BstNBI; Nt.CviPII. The mixture was incubated at 37 degrees for one hour.

After the nicking reaction completed, the experiment proceeded with limited polymerase extension at the nicking sites to displace the 3′ down stream strand and form a single stranded flap. The flap generation reaction mix consisted of 15 μl of nicking product and 5 μl of incorporation mix containing 2 μl of 10× buffer, 0.5 μl of polymerase including but not limited to vent(exon-), and 1 μl nucleotides at various concentration from 1 uM to 1 mM. The flap generation reaction mixture was incubated at 55 degrees. The length of the flap was controlled by the incubation time, the polymerases employed and the amount of nucleotides used. The generated flaps were then hybridized and labeled with universal probes such as TGAGGCAGGAGAAT (SEQ ID NO: 9) for Nb.BbVCI.

### Example

### Structural Variation Validation of Rearranged Structure of MCF-7 3F5 BAC Clone from the Breast Cancer Genome

This region consists of four segments: 3p14.1, an inverted 14.1 Kb block; 20q12, an inverted 22.3 Kb block containing exon 6 of the PTPRT gene; 20p13.31, a 45.5 Kb block containing exon 1 of the truncated BMP7 gene along with its intact promoter; 20p13.2, a 23.4 Kb block containing the complete ZNF217 gene. Region specific probes hybridized to the flaps are used to confirm the presence of the four regions, TGCCACCTACCCCT (SEQ ID NO: 5) for 20q12; AGAAGCCTGTCAGATGCAT (SEQ ID NO: 6) for 20p13.31; ACTGTAGTCTTGAATTCCTGA (SEQ ID NO: 7) for 20p13.2 and TCCTTGGTTGACCTAACAACACA (SEQ ID NO: 8) for 3p14.1.

### Example

### Detection Schemes

In one example of a detection scheme, video images of DNA moving in flow mode are captured by a time delay and integration (TDI) camera. In such an embodiment, the movement of the DNA is synchronized with the TDI.

In another example of a detection scheme, video images of a DNA moving in flow mode are capture by a CCD or CMOS camera, and the frames are integrated by software or hardware to identify and reconstruct the image of the DNA.

In another example of a detection scheme, video images of a DNA are collected by simultaneously capturing different wavelengths on a separate set of sensors. This can be done using one camera and a dual or multi-view splitter, or using filters and multiple cameras. The camera can be a TDI, CCD or CMOS detection system.

In another example, using simultaneous multiple wavelength video detection, the backbone dye is used to identify a unique DNA fragment, and the labels are used as markers to follow the DNA movement. This is useful for when the length of the DNA is greater than the field of view of the camera, and the markers can serve to help map a reconstructed image of the DNA.

1.-39. (canceled)

40. A method for generating a pattern of sequence-related structural features of a double stranded DNA, comprising:
nicking one strand of a double stranded DNA at a nick site by an agent capable of introducing a single stranded break;
labeling the nicked DNA at or about the nick site;
ligating the labeled DNA with a ligase; and
detecting the label on the labeled DNA to generate a pattern of sequence-related structural features of the double stranded DNA.

41. The method of claim 40, wherein said nicking is accomplished with a site-specific nicking enzyme.

42. The method of claim 41, wherein said nicking, labeling, ligating, and detecting are each performed at multiple sites on the DNA.

43. The method of claim 42, further comprising transporting the ligated DNA into a nanochannel and maintaining the DNA in elongated form in the nanochannel.

44. The method of claim 40, wherein the label is fluorescent.

45. The method of claim 40, wherein the label is a fluorescently-labeled base.

46. The method of claim 40, wherein after said nicking the DNA has a break in a single strand, into which at least one nucleotide is introduced.

47. The method of claim 46, wherein said nick separates first and second pieces of the nicked strand and wherein prior to said ligating said at least one nucleotide is joined to said first piece but not to said second piece.

48. The method of claim 46, wherein said at least one nucleotide is labeled.

49. The method of claim 48, further comprising transporting the labeled DNA into a nanochannel prior to the detecting step.

50. The method of claim 40, further comprising:
generating a DNA flap at the nick site from the nicked strand; and
removing the flap prior to the ligation step.

51. A method for generating a pattern of sequence-related structural features of a double-stranded DNA, comprising:
nicking the double-stranded DNA with a site-specific nicking enzyme without breaking the other strand;
incorporating one or more bases into the nicking site of the nicked DNA, wherein incorporating the bases comprises contacting the nicked DNA with:
a. a polymerase;
b. one or more nucleotides; and
c. a ligase.
wherein at least one said nucleotide is labeled, thus labeling the DNA; and
detecting the label on the labeled DNA to generate a pattern of sequence-related structural features of the DNA.

52. The method of claim 51, wherein said nicking, incorporating, and detecting are each performed at multiple sites on the DNA.

53. The method of claim 52, further comprising transporting the ligated DNA into a nanochannel and maintaining the DNA in elongated form in the nanochannel, wherein the nannochannel has a cross sectional area of about 1 to about 10⁶ square nanometers.

54. The method of claim 51, wherein the label is fluorescent.

55. The method of claim 52, wherein a pattern of said labels is detected, further comprising:
correlating the detected pattern with a characteristic of the DNA.

56. The method of claim 55, wherein the characteristic of the DNA is a sequence characteristic.

57. The method of claim 53, wherein the nanochannel has an inner diameter of less than 500 nm.

58. The method of claim 40, wherein the structural features comprises DNA sequence, haplotype, DNA structural variations, DNA copy number, presence or absence of a portion of a pathogen genomic DNA, or any combination thereof.

59. The method of claim 40, wherein the agent capable of introducing a single stranded break is a nickase, a nicking endonucleoase, an electromagnetic wave, free radicals, or any combination thereof.


SEQUENCE LISTING (9 sequences)

SEQ ID NO: 1 - DNA, 30 bp, Artificial Sequence
  Synthetic oligonucleotide
tccaactatataatttgaccagagaacaag

SEQ ID NO: 2 - DNA, 20 bp, Artificial Sequence
  Synthetic oligonucleotide
aaggtcttgagcaggccgtt

SEQ ID NO: 3 - DNA, 50 bp, Artificial Sequence
  Synthetic oligonucleotide
tgaggcaggagaatcacttgaacccaggaggcggaggttgcagtgagccg

SEQ ID NO: 4 - DNA, 14 bp, Artificial Sequence
  Synthetic oligonucleotide
tgaggcaggagaat

SEQ ID NO: 5 - DNA, 14 bp, Artificial Sequence
  Synthetic oligonucleotide
tgccacctacccct

SEQ ID NO: 6 - DNA, 19 bp, Artificial Sequence
  Synthetic oligonucleotide
agaagcctgtcagatgcat

SEQ ID NO: 7 - DNA, 21 bp, Artificial Sequence
  Synthetic oligonucleotide
actgtagtcttgaattcctga

SEQ ID NO: 8 - DNA, 23 bp, Artificial Sequence
  Synthetic oligonucleotide
tccttggttgacctaacaacaca

SEQ ID NO: 9 - DNA, 14 bp, Artificial Sequence
  Synthetic oligonucleotide
tgaggcaggagaat